(19) United States
(12) Patent Application Publication
(10) Pub. No.: US 2025/0092314 A1
(43) Pub. Date: Mar. 20, 2025

(54) Pyrolysis plant and method for thermal mineralization of biomass and production of combustible gases, liquids and biochar

(71) Applicant: Frichs Holding 2 ApS, Horsens (DK)

(72) Inventor: Ove Kudahl MUNCH, Tilst (DK)

(21) Appl. No.: 18/964,936

(22) Filed: Dec. 2, 2024

Related U.S. Application Data
(63) Continuation of application No. PCT/DK2023/050129, filed on May 25, 2023.

(30) Foreign Application Priority Data
Jun. 3, 2022 (DK) PA 2022 00527

(51) Int. Cl. C10B 53/02; B01D 53/14; C10J 1/207; C25B 15/08

(52) U.S. Cl. CPC C10B 53/02; B01D 53/1475; C10J 1/207; C25B 15/081; B01D 2257/504; C10J 2200/152

(57) ABSTRACT

A pyrolysis plant comprising a reactor for producing pyrolysis gas from biomass is disclosed. The reactor comprises one or more reaction channels thermally connected to at least one heating circuit, which is configured to heat the reaction channels to a temperature that is high enough to gasify the biomass, where the reactor comprises a feed section configured for feeding the biomass into the reaction channels. Each reaction channel constitutes a heating circuit integrated in the reaction channel, wherein the heating circuit comprises a gas mixture unit and a plurality of input nozzles arranged and configured to introduce a mix of oxygen and CO₂ from the gas mixture unit into the reaction channel.

### CROSS-REFERENCE TO RELATED APPLICATIONS

This application is a continuation under 35 U.S.C. 111 of International Patent Application No. PCT/DK2023/050129, filed May 25, 2023, which claims the benefit of and priority to Danish Application No. PA 2022 00527, filed Jun. 3, 2022, each of which is hereby incorporated by reference in its entirety.

### FIELD OF INVENTION

The present invention relates to a pyrolysis plant and method for thermal mineralization of biomass and production of combustible gases, liquids and biochar. The present invention also relates to a plant comprising such pyrolysis plant and an additional plant.

### BACKGROUND

Pyrolysis is a well-known process, which is used for converting organic materials into energy in the form of gas. Many methods and reactor designs have been developed over the course of time.

Pyrolysis makes it possible to convert biomass such as straw, farmyard manure, energy crops or organic residues, to a gas, which can be used for example in a combined heat and power station. The ash from the process is rich in nutrients that are required for growth and development of crops.

In a typical pyrolysis plant, comminuted biomass is fed into a pyrolysis chamber, which is heated in the absence of oxygen. As no oxygen is present, the biomass does not burn. Instead, the biomass is converted to approx. 80% pyrolysis gas and 20% coke (carbon). Sand particles are injected from the bottom of the pyrolysis chamber, for the purpose of swirling the coke particles and entraining them out of the pyrolysis chamber. The pyrolysis gas formed and the coke are withdrawn from the upper part of the pyrolysis chamber and transferred to a first cyclone, where the sand and coke particles are separated and go down into a coke reactor, while the pyrolysis gases are transferred to another cyclone, where the ash, which contains nutrient salts, is separated and is transferred to a container. The gases leaving the other cyclone can now be used in for example combined heat and power stations.

The coke reactor is configured for gasifying the coke. The gas is led to the pyrolysis chamber. Air is fed into the coke reactor.

In a prior art pyrolysis apparatus for rapid conversion of petrochemical-based waste to gas and liquid fuel, biomass is sent through an external reaction channel consisting of one or more tubes. The tubes are arranged and configured to be heated via heat transfer between the walls of the tubes and one or more adjacent heating circuits. Accordingly, the thickness of the walls separating the tubes and one or more adjacent heating circuits impacts the conversion process. It has been experimentally observed that the rate of heat conduction through a layer is proportional to the temperature difference across the layer and the heat transfer area, but it is inversely proportional to the thickness of the layer. Accordingly, the thickness of the walls is a main determinant of the rate of heat conduction.

Since the rate of heat conduction determines how fast heat can be transferred to the biomass in the reaction channel, it is a disadvantage to apply thick walls.

It is desirable to increase the speed by which heat can be transferred to the biomass in the heated vessel. Thus, it is an object of the present disclosure to provide a pyrolysis plant for thermal mineralization of biomass and production of combustible gases, liquids and biochar, in which pyrolysis plant the speed by which heat can be transferred to the biomass in the reaction channel can be increased. It is also an object to provide a method for thermal mineralization of biomass and production of combustible gases, liquids and biochar, which method allows for increasing the speed by which heat can be transferred to the biomass in the reaction channel.

US20130195727 A1 discloses a fluidized bed biogasifier for gasifying biosolids. The biogasifier includes a reactor vessel and a feeder for feeding biosolids into the reactor vessel at a desired feed rate during steady-state operation of the biogasifier. A fluidized bed in the base of the reactor vessel has a cross-sectional area that is proportional to at least the fuel feed rate such that the superficial velocity of gas is in the range of 0.1 m/s to 3 m/s. The temperature within the gasifier is controlled by introducing ambient air. The oxygen in the ambient air is hereby used to heat the reactor vessel. By introducing ambient air into the gasifier, a large quantity of nitrogen gas (N₂) enters the gasifier. This is a huge disadvantage because the N₂ would have to be removed from the pyrolysis gas. Accordingly, it would be desirable to have an alternative solution.

US20130195727 A1 discloses a fluidized bed biogasifier for gasifying biosolids. The biogasifier includes a reactor vessel and a feeder for feeding biosolids into the reactor vessel at a desired feed rate during steady-state operation of the biogasifier. A fluidized bed in the base of the reactor vessel has a cross-sectional area that is proportional to at least the fuel feed rate such that the superficial velocity of gas is in the range of 0.1 m/s (0.33 ft/s) to 3 m/s (9.84 ft/s). In a method for gasifying biosolids, biosolids are fed into a fluidized bed reactor. Oxidant gases are applied to the fluidized bed reactor to produce a superficial velocity of producer gas in the range of 0.1 m/s (0.33 ft/s) to 3 m/s (9.84 ft/s). The biosolids are heated inside the fluidized bed reactor to a temperature range between 900° F. (482.2° C.) and 1700° F. (926.7° C.) in an oxygen-starved environment having a sub-stoichiometric oxygen level, whereby the biosolids are gasified. It would be desirable to be able to increase the efficiency of the system.

US20130025200 A1 discloses a gasifier system for converting biomass to biogas. The system comprises a reaction chamber with a biomass supply port for receiving a biomass volume, a waste outlet port for discharging biomass conversion by-products, a gas inlet for receiving heated oxidizing gas, a gas outlet for discharging generated biogas and a burner manifold for distributing oxidizing gas within the chamber to react the biomass. The burner manifold includes primary tubes and secondary tubes, positioned in a vertically lower part of the chamber and configured with multiple openings or ports for dispensing the oxidizing gas, where the secondary tubes extend into, inject and evenly distribute the oxidizing gas into the biomass volume to optimize conversion to biogas. It would be desirable to be able to increase the efficiency of the system.

### BRIEF DESCRIPTION

A pyrolysis plant according to the present disclosure is a pyrolysis plant comprising a reactor for producing pyrolysis gas from biomass, wherein the reactor comprises at least one reaction channel and at least one heating circuit, which is configured to heat the at least one reaction channel to a temperature that is high enough to gasify the biomass, where the reactor comprises a feed section configured for feeding the biomass into the at least one reaction channel, wherein the reaction channel constitutes the heating circuit being integrated in the reaction channel, wherein the heating circuit comprises:
a gas mixture unit and a plurality of input nozzles arranged and configured to introduce a mix of only oxygen and CO₂ from the gas mixture unit into the reaction channel, wherein the pyrolysis plant comprises at least one gas sensor arranged and configured to detect the concentration of oxygen (O₂) in the heating circuit, wherein the pyrolysis plant comprises a control unit and one or more temperature sensors, wherein the one or more temperature sensors are arranged and configured to measure the temperature in the reaction channel, wherein the control unit is arranged and configured to regulate the flow and/or oxygen concentration of the mix of oxygen and CO₂ into the reaction channel in dependency of the temperature in the reaction channel.

Hereby, it is possible to maintain the temperature in the reaction channel within a predefined temperature range in order to optimize the efficiency of the pyrolysis plant.

The disclosed systems and methods make it possible to increase the speed by which heat can be transferred to the biomass in the reaction channel. Moreover, it is possible to provide a simple construction since a single circuit is used as both reaction channel and heating circuit. Heat is generated inside the reaction channel and this heat does not need to pass through a wall to enter the reaction channel like in the prior art.

The pyrolysis plant is configured for producing pyrolysis gas from biomass such as e.g. straw, wood chips, farmyard manure, energy crops or other products that contain carbon and hydrogen.

The reactor comprises at least one reaction channel and at least one heating circuit, which is configured to heat the at least one reaction channel to a temperature that is high enough to gasify the biomass.

The reactor comprises a feed section configured for feeding the biomass into the at least one reaction channel. It is typical for the feed section to be configured to limit the supply of oxygen, so that the oxygen concentration in the gas that is fed into the at least one reaction channel is far lower than the oxygen concentration in the atmospheric air.

In an embodiment, the pyrolysis plant comprises a gas accelerator configured for providing a gas flow velocity that is able to blow the biomass around in the reaction channels.

Distribution of biomass in the at least one reaction channel may be provided by using a blower (e.g. an electric blower, where the motor is equipped with a frequency converter). The gas accelerator may thus be a blower.

The gas accelerator may consist of a mechanical device, which for example comprises a fan.

It may be advantageous if the heating circuit is configured to carry out heating by gas burning.

The reaction channel constitutes the heating circuit being integrated in the reaction channel, wherein the heating circuit comprises:
a gas mixture unit and a plurality of input nozzles arranged and configured to introduce a mix of only oxygen and CO₂ from the gas mixture unit into the reaction channel.

The reaction channel and the heating circuit are a single circuit.

It is an advantage that the inactive gas is CO₂ that can be stripped out again.

It may be advantageous to have a large number of nozzles in order to distribute the mix of oxygen and CO₂ evenly along the length of the reaction channel and to avoid large local oxygen concentrations.

It may be advantageous if the pyrolysis chamber comprises at least one flow sensor that is arranged and configured to measure a flow in the reaction channel.

Oxygen is introduced to generate heat. Accordingly, the regulation of the oxygen content in the mixed gas is of great importance. In an embodiment, the oxygen concentration in the heating circuit is detected in order to enable a regulation of the heat generation process.

In an embodiment, one or more gas sensors suitable for detecting the oxygen concentration are arranged in the reaction circuit. Since all oxygen should have been used during the heat generation process, the oxygen concentration should be zero or very close to zero. Therefore, gas sensors suitable for detecting oxygen concentration in the reaction circuit will in practice primarily be used as a safety device.

In an embodiment, the pyrolysis plant comprises a plurality of temperature sensors arranged and configured to measure the temperature in the reaction channel.

In an embodiment, the control unit is configured to compare the temperature in the reaction channel with a predefined temperature interval.

In an embodiment, the control unit is configured to reduce the flow and/or oxygen concentration of mixed gas introduced into the reaction channel if the temperature in the reaction channel is above the predefined temperature interval.

In an embodiment, the control unit is configured to increase the flow and/or concentration of oxygen of the mixed gas introduced into the reaction channel if the temperature in the reaction channel is below the predefined temperature interval.

Regulation of the oxygen content of the mixed gas can be regulated by:
a) changing the flow of the mixed gas; and/or
b) changing the oxygen concentration of the mixed gas.

In an embodiment, the pyrolysis plant comprises a regulation unit configured to regulate the oxygen content of the mixed gas on the basis of measurements of the oxygen concentration and/or flow of the mixed gas.

In an embodiment, the pyrolysis plant comprises a heating unit arranged and configured to heat the mix of oxygen and CO₂ before the mix enters the reaction channel.

In an embodiment, the pyrolysis plant comprises an outlet for evacuating gas out from the reaction channel. The gas can be processed in an external CO₂ stripping device arranged and configured to move CO₂ out of the gas removed from the reaction channel.

In an embodiment, the production plant is a production plant comprising a pyrolysis plant and an electrolyzer, wherein the electrolyzer is connected to the pyrolysis plant in a manner in which oxygen (O₂) from the electrolyzer is provided to the pyrolysis plant via an oxygen inlet.

In an embodiment, the production plant comprises a Power-to-Gas or Power-to-Liquid plant, wherein the Power-to-Gas or Power-to-Liquid plant is connected to and receives CO₂ that is stripped from gas from the reaction channel of the pyrolysis plant.

In an embodiment, a production plant comprises a pipe for conducting hydrogen from an electrolyzer to the Power-to-Gas or Power-to-Liquid plant.

A method according to the present disclosure is a method for producing pyrolysis gas from biomass in a pyrolysis plant comprising a reactor for producing pyrolysis gas from biomass, where the reactor comprises at least one reaction channel and at least one heating circuit, which is configured to heat the at least one reaction channel to a temperature that is high enough to gasify the biomass, wherein the reactor comprises a feed section configured for feeding the biomass into the at least one reaction channel, wherein the reaction channel constitutes the heating circuit being integrated in the reaction channel, wherein the method comprises the following steps:
heating the reaction channel by introducing a mix of only oxygen and CO₂ in into the reaction channel;
detecting the concentration of oxygen (O₂) in the heating circuit;
detecting the temperature (T) in the reaction channel; and
regulating the flow of the mix of oxygen and CO₂ into the reaction channel in dependency of the temperature (T) in the reaction channel.

Detecting the concentration of oxygen (O₂) in the heating circuit may be done by one or more gas sensors arranged and configured to detect the concentration of oxygen (O₂) in the reaction channel.

In an embodiment, the method applies a gas mixture unit and a plurality of input nozzles that are arranged and configured to introduce a mix of oxygen and CO₂ from the gas mixture unit into the reaction channel.

It is an advantage that the inactive gas is CO₂.

In an embodiment, the pyrolysis plant comprises a plurality of temperature sensors arranged and configured to measure the temperature in the reaction channel.

In an embodiment, the method comprises the step of:
a) comparing the temperature (T) in the reaction channel with a predefined temperature interval.

In an embodiment, the method comprises the step of:
b) reducing the flow of mixed gas introduced into the reaction channel if the temperature (T) in the reaction channel is above the predefined temperature interval.

In an embodiment, the method comprises the step of:
c) increasing the flow and/or the concentration of oxygen of the mixed gas introduced into the reaction channel if the temperature (T) in the reaction channel is below the predefined temperature interval.

In an embodiment, the method comprises the step of heating the mix of oxygen and the CO₂ before the mix enters the reaction channel.

In an embodiment, the method comprises the step of moving the CO₂ out of gas from the reaction channel.

In an embodiment, the CO₂ is removed by a CO₂ stripping device (e.g. an external CO₂ stripping device).

### BRIEF DESCRIPTION OF THE DRAWINGS

The present systems and methods will become more fully understood from the detailed description given herein below. The accompanying drawings are given by way of illustration only, and thus, they are not limitative. In the accompanying drawings:

FIG. 1A shows a schematic view of a portion of a reactor according to an embodiment;

FIG. 1B shows a schematic view of a portion of a reactor according to an embodiment;

FIG. 2A shows a mixing unit of a production plant according to an embodiment;

FIG. 2B shows a graph depicting the flow of a mixture of oxygen and CO₂ as a function of time;

FIG. 2C shows a graph depicting the temperature inside the reactor as a function of time;

FIG. 3 shows a flowchart illustrating a process used to regulate the flow of mixed gas introduced into the reaction channel;

FIG. 4A shows a schematic view of a portion of a prior art pyrolysis plant reactor;

FIG. 4B shows a close-up view (sectional view) of a part of a reactor corresponding to the reactor shown in FIG. 4A;

FIG. 5A shows a schematic illustration of a biomass feed unit 30 for introducing biomass into a reactor of a pyrolysis plant according to an embodiment; and

FIG. 5B shows a pyrolysis plant according to an embodiment comprising an electrolyzer.

### DETAILED DESCRIPTION

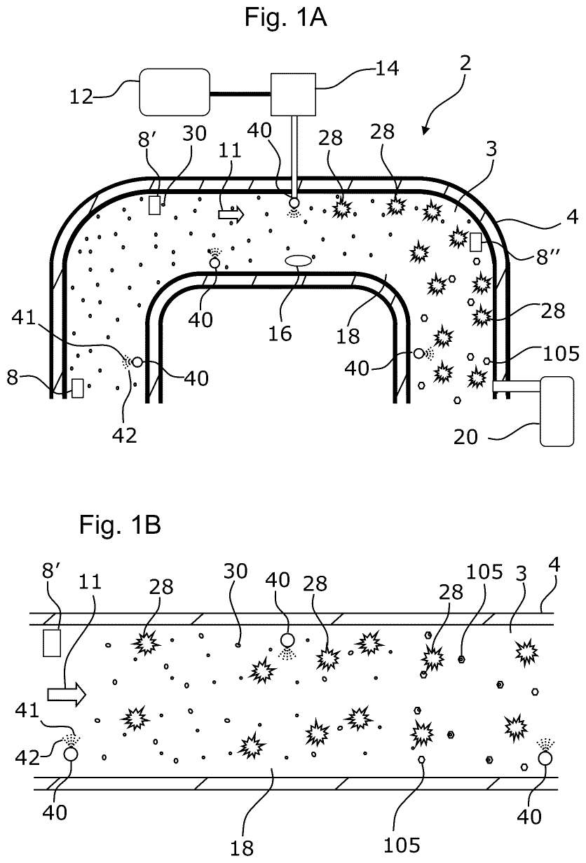

Referring now in detail to the drawings for the purpose of illustrating embodiments of the present systems and methods, a reactor 2 is illustrated in FIG. 1A.

FIG. 1A illustrates a schematic view of a portion of a reactor 2 according to an embodiment. The reactor 2 comprises a reaction channel 3 that constitutes a heating circuit 18. It should be noted that FIG. 1A is a schematic view only. Accordingly, the reactor 2 may have a different geometry.

In an embodiment, the reactor 2 only comprises one reaction channel 3. In an embodiment, the reactor 2 comprises several reaction channels 3.

The biomass 30 is fed into the reaction channel 3 of the reactor 2 in a section that contains a carrier gas, which carrier gas is recirculated in the reaction channel 3. In an embodiment, the carrier gas is the pyrolysis gas 28 produced in the reaction channel 3. In an embodiment, when more and more biomass 30 is gradually gasified, the increased pressure of the pyrolysis gas 28 in the reaction channel 3 will force a portion of the pyrolysis gas 28 to leave the reaction channel 3 (e.g. through an ejection process). The biomass 30 will normally be comminuted before feeding it into the reaction channel 3.

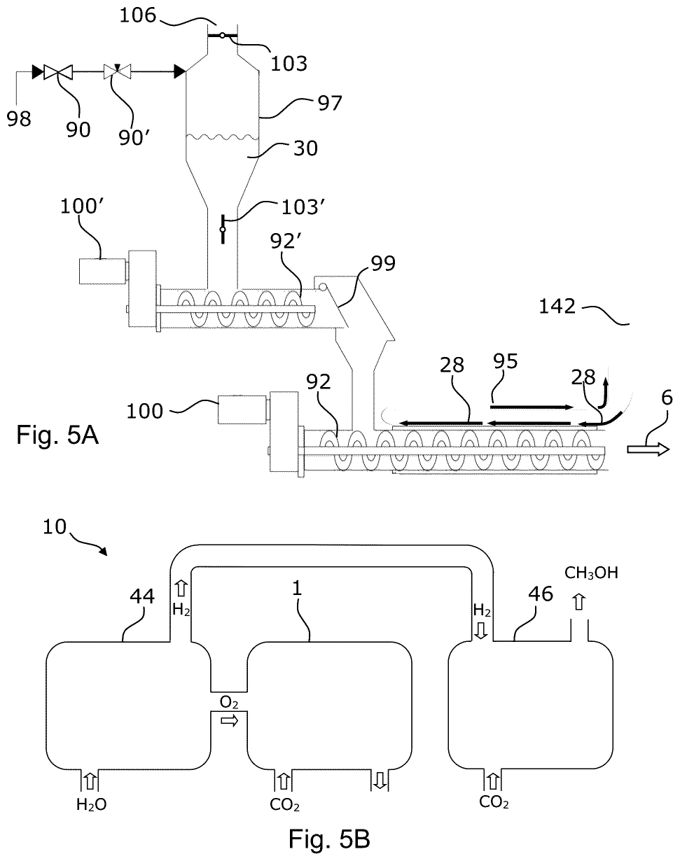

Feed of biomass 30 may be carried out by a metering screw or a feed screw (see FIG. 5). Recirculation of the carrier gas can be provided by a gas accelerator, which may for example be configured as a blower. In an embodiment, the gas accelerator is placed inside the reaction channel 3. The gas accelerator should be arranged and configured to generate a pressure gradient and therefore a non-zero gas flow velocity 11.

A non-zero gas flow velocity makes it possible to maintain recirculation of the carrier gas. A non-zero gas flow velocity also ensures that the biomass 30 is being distributed in the reaction channel 3 of the reactor. In the reaction channel 3, the biomass 30 is gasified and forms pyrolysis gas 28. Accordingly, the reaction channel 3 constitutes the pyrolysis chamber of the reactor 2. However, as mentioned earlier, the reaction channel 3 also constitutes the heating circuit 18 of the reactor 2.

The reactor 2 is configured to heat the biomass 30 in a faster manner than conventional pyrolysis plants, in which the biomass is introduced with a screw and then lies in a relatively thick layer. As the biomass in conventional installations is introduced in a manner in which a relatively thick layer of biomass forms on the reactor bottom, the heating of the biomass does not take place uniformly (as the biomass has an insulating effect and therefore it is far colder in the middle of the layer than in the uppermost part of the layer). Due to this temperature gradient, moreover, the heating time is relatively long compared to the heating time in a reactor 2 according to the present disclosure. Accordingly, the heating of the biomass 30 happens in a faster and much more even manner in a reactor 2 according to the present disclosure than in a conventional pyrolysis plant.

The heating circuit 18 comprises a plurality of nozzles 40 arranged and configured to introduce a mixture of oxygen 41 and CO₂ 42 into the reaction channel 3. By applying nozzles 40 that are configured to supply a mixture of oxygen 41 and CO₂ 42 to the heating circuit 18, it is possible to both control the amount of gas (mixture of oxygen 41 and CO₂ 42) that is fed into the heating circuit 18 and provide a desired distribution of the gas (mixture of oxygen 41 and CO₂ 42).

It may be advantageous that the nozzles 40 are arranged in a configuration, in which the gas (mixture of oxygen 41 and CO₂ 42) is evenly distributed along one or more feed zones (corresponding to the placement of the nozzles). In this way, it is possible to avoid local overheating (hot spots).

In an embodiment, the nozzles 40 are arranged in a configuration in which the distance between adjacent nozzles 40 is in a range of 50-200 cm.

In an embodiment, all the nozzles 40 are configured for introducing gas simultaneously. In an embodiment, all the nozzles 40 are configured for introducing gas with the same flow (feed rate).

On the left side of the section of the reaction channel 3 shown in FIG. 1A the concentration of biomass 30 is relatively high. On the right side of the section of reaction channel 3 shown in FIG. 1A there is a lower concentration of biomass 30, whereas the concentration of pyrolysis gas 28 and biochar (carbon) 105 is higher. The reason for this is that the biomass 30 has been converted to pyrolysis gas 28 and biochar (carbon) 105, respectively.

The reactor 2 comprises a plurality of temperature sensors 8, 8′, 8″ that are arranged to detect the temperature inside the reaction channel 3.

The reactor 2 is part of a pyrolysis plant that comprises a control unit 12 and a heating unit 14. The heating unit is arranged and configured to heat the mixture of oxygen 41 and CO₂ 42 before the nozzles 40 introduce the mixture of oxygen 41 and CO₂ gas 42 into the reaction channel 3.

A gas sensor 16 is arranged in the reaction channel 3. The gas sensor 16 is arranged and configured to detect the concentration of one or more gasses inside the reaction channel 3. In an embodiment, the gas sensor 16 is arranged and configured to detect the concentration of oxygen inside the reaction channel 3.

The control unit 12 is arranged and configured to regulate the flow and/or oxygen concentration of the mix of oxygen 41 and CO₂ 42 into the reaction channel 3 in dependency of the temperature in the reaction channel 3.

In an embodiment, the control unit 12 is configured to:
a) compare the temperature (detected by the temperature sensors 8, 8′, 8″) in the reaction channel 3 with a predefined temperature interval,
b) reduce the flow of mixed gas introduced into the reaction channel 3 if the temperature in the reaction channel 3 is above the predefined temperature interval,
c) increase the flow and/or the concentration of oxygen of the mixed gas 41, 42 introduced into the reaction channel 3 if the temperature in the reaction channel 3 is below the predefined temperature interval.

The pyrolysis plant comprises a heating unit 14 of the mix of oxygen 41 and CO₂ 42 before it enters the reaction channel 3.

In an embodiment, the pyrolysis plant comprises an outlet 20 for evacuating gas. The gas can be processed in an external CO₂ stripping device (not shown) arranged and configured to move CO₂ out of the gas removed from the reaction channel.

FIG. 1B illustrates a schematic view of a portion of a reactor 2 according to the present disclosure. The reactor 2 comprises a reaction channel 3 that constitutes a heating circuit 18. The reactor 2 basically corresponds to the one shown in and explained with reference to FIG. 1A.

The reactor 2 is configured to receive biomass 30 that is fed into the reaction channel 3 of the reactor 2 in a section that contains a carrier gas, which carrier gas is recirculated in the reaction channel 3. The gas flow velocity 11 is indicated. The gas flow causes recirculation of the carrier gas and ensures that the biomass 30 is being distributed in the reaction channel 3 of the reactor. The biomass 30 is gasified and forms pyrolysis gas 28 in the reaction channel 3. A temperature sensor 8′ is arranged in the reaction channel 3. The temperature sensor 8′ is configured to detect the temperature inside the reaction channel 3.

The heating circuit 18 comprises several nozzles 40 arranged and configured to introduce a mixture of oxygen 41 and CO₂ 42 into the reaction channel 3.

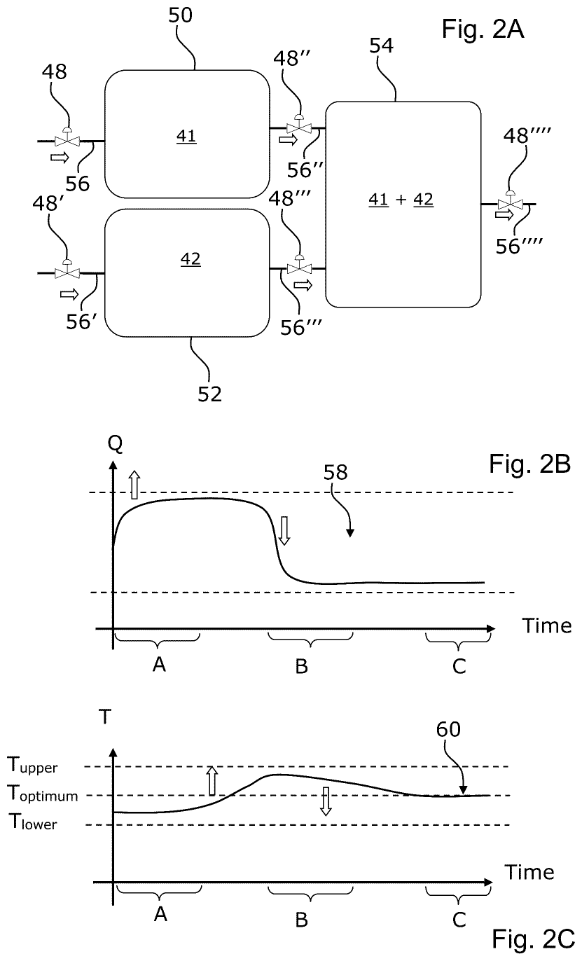

FIG. 2A illustrates a mixing unit of a pyrolysis plant according to the present disclosure. The mixing unit comprises a mixing chamber 54 provided with a pipe 56″ designed as an outlet that is configured to be connected to nozzles arranged and configured to introduce the mix of oxygen 41 and CO₂ into the reaction channel of a pyrolysis plant according to the present disclosure.

The mixing unit is configured to receive oxygen 41 from a tank 50 that is connected to the mixing chamber 54 via a pipe 56″. The mixing unit is configured to receive CO₂ 42 (e.g. CO₂) from a tank 52 that is connected to the mixing chamber 54 via a pipe 56′″. The oxygen containing tank 50 comprises an inlet pipe 56. Likewise, the CO₂ containing tank 52 comprises an inlet pipe 56′.

A valve 48, 48′, 48″, 48′″, 48″″ is provided on each pipe 56, 56′, 56″, 56′″, 56″″ in order to allow for decreasing the flow through the respective pipe. In an embodiment, at least some of the valves 48, 48′, 48″, 48′″, 48″″ are remote control valves.

In an embodiment, the percentage of oxygen 41 in the tank 54 is in the range 5-10 vol %.

FIG. 2B illustrates a graph 58 depicting the flow of a mixture of oxygen and CO₂ as a function of time. FIG. 2C illustrates a graph 60 depicting the temperature inside the reactor as a function of time. It can be seen that in the first time period A, the temperature inside the reactor is above a predefined lower temperature T_{lower} but below a predefined optimum temperature Tₒₚₜᵢₘᵤₘ. Accordingly, in order to increase the temperature inside the reactor, the flow Q of the mixture of oxygen and CO₂ is increased (indicated with an arrow that points upwards). Due to the increased flow Q of the mixture of oxygen and CO₂, the temperature increases.

It can be seen that in a second time period B, the temperature inside the reactor approaches a predefined upper temperature Tᵤₚₚₑᵣ. Accordingly, in order to prevent the temperature inside the reactor from exceeding the upper temperature Tᵤₚₚₑᵣ, the flow Q of the mixture of oxygen and CO₂ is decreased (indicated with an arrow that points downwards). Due to the decreased flow Q of the mixture of oxygen and CO₂, the temperature decreases.

It can be seen that in a third time period C, the temperature inside the reactor approaches the predefined optimum temperature Tₒₚₜᵢₘᵤₘ. Accordingly, the flow level is kept steady.

The flow of a mixture of oxygen and CO₂ is regulated on the basis of the detected temperature inside the reactor.

It is typical that at least one gas sensor designed to detect the oxygen concentration is arranged in the tank 54. Hereby, it is possible to monitor the oxygen concentration in the tank 54 and regulate (e.g. increase) the temperature by changing (e.g. increasing) the oxygen concentration in the tank. It is important to minimize the quantity of CO₂ being introduced into the tank 54. Therefore, the control of the amount of oxygen in the tank 54 is important.

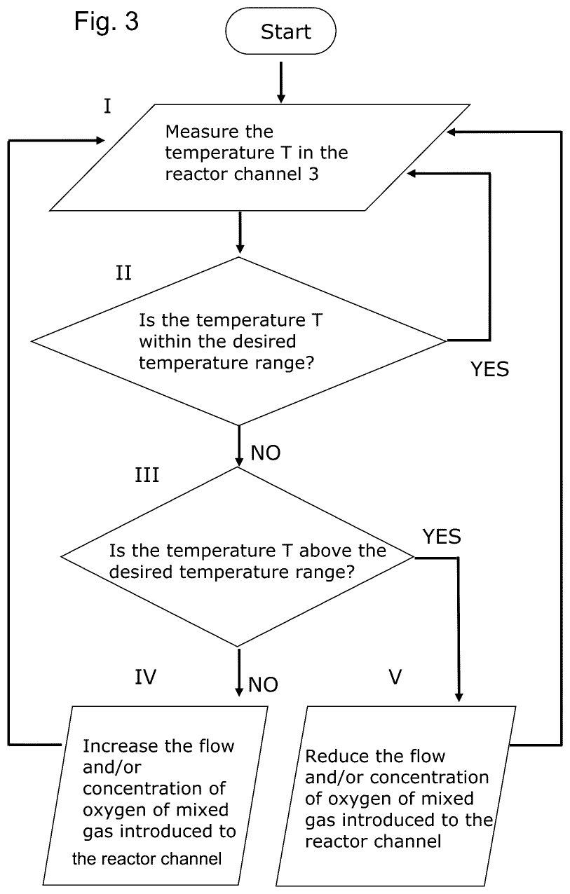

FIG. 3 shows a flowchart illustrating a process used to regulate the flow of mixed gas introduced into the reaction channel 3. In the first step I the temperature T in the reaction channel 3 is measured. The temperature T can be measured by one or more temperature sensors. In an embodiment, the pyrolysis plant comprises a plurality of temperature sensors arranged and configured to measure the temperature in the reaction channel.

In the second step II the temperature T in the reaction channel 3 is compared with a predefined temperature interval. If the detected temperature T is within the predefined temperature interval, the first step I is repeated. If the detected temperature T is not within the predefined temperature interval, a third step III is carried out. In an embodiment, the predefined temperature interval is defined by a first low temperature and a second higher temperature.

In the third step III it is determined if the temperature T in the reaction channel 3 is above the predefined temperature interval. If the temperature T in the reaction channel 3 is above the predefined temperature interval, a fifth step V is carried out. In the fifth step V the flow of mixed gas introduced into the reaction channel is reduced. An example of such flow reduction is shown in and explained with reference to FIG. 2B. When the fifth step V has been carried out, the first step I is carried out again.

On the other hand, if the temperature T in the reaction channel 3 is below the predefined temperature interval, a fourth step IV is carried out. In the fourth step IV the flow and/or the concentration of oxygen of the mixed gas (oxygen and CO₂) introduced into the reaction channel is increased. An example of such flow increasement is shown in and explained with reference to FIG. 2B. When the fourth step IV has been carried out, the first step I is carried out again.

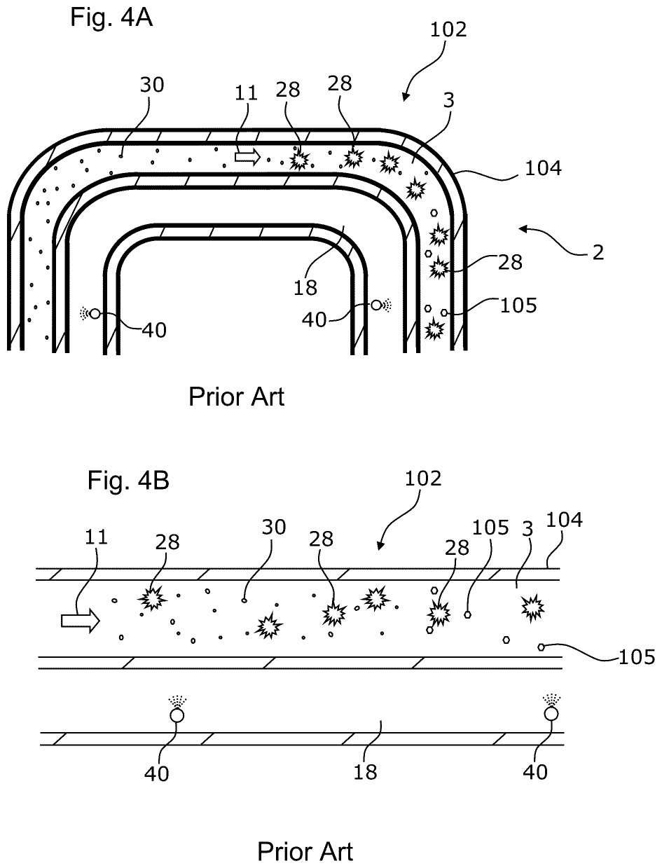

FIG. 4A illustrates a schematic view of a portion of a prior art pyrolysis plant reactor 102. The reactor 102 comprises a reaction channel 3, which is placed in a heat exchanger 104 designed to exchange heat with the surrounding heating circuit 18. Biomass 30 is fed into reaction channel 3 in a section that contains a carrier gas, which is recirculated through the reaction channel 3.

The heating circuit 18 is provided with nozzles 40, which are configured for supplying gas to the heating circuit 18. Hereby, it is possible to control the amount of gas that is fed into the heating circuit 18. The nozzles 40 supply pyrolysis gas 28 that is produced in the reaction channel 3.

On the left side of the section of the reaction channel 3 shown, there is a relatively high concentration of biomass 30. On the right side of the section of reaction channel 3 shown, there is on the other hand a lower concentration of biomass 30, while conversely there is a higher concentration of pyrolysis gas 28 and biochar (carbon) 105 because the biomass 30 has been converted to pyrolysis gas 28 and biochar (carbon) 105, respectively.

FIG. 4B illustrates a close-up view (sectional view) of a part of a reactor corresponding to the reactor shown in FIG. 4A. The reactor comprises a heat exchanger 104, which is in thermal contact with an adjacent heating circuit 18 provided with a channel that extends parallel to the heat exchanger 104. Biomass 30 is fed into the reaction channel 3. The biomass 30 is gasified when a sufficiently high temperature (typically above 800° C.) is provided, and at the same time the oxygen content is kept low.

FIG. 5A illustrates a schematic illustration of a biomass feed unit for introducing biomass 30 into a reactor of a pyrolysis plant. The purpose of the biomass feed unit is to control the concentration of atmospheric air that is present in the biomass 30 that is fed into the reactor. It is an advantage to minimize the amount of nitrogen from the atmospheric air that is fed into the reactor. A silo 97 is provided, equipped with an upper inlet 106, which in normal conditions is kept closed with a valve 103. This valve 103 is configured to be brought into an open configuration when biomass 30 is filled in the silo 97.

An outlet is provided in the lower part of the silo 97. Under normal conditions the outlet is kept open by a valve 103′. This valve 103′ is configured to shut off the outlet when biomass 30 is filled in the silo 97.

In an embodiment, a sensor (not shown) is arranged and configured to measure the amount of biomass 30 in the silo 97. Measurements from this sensor may be applied to control when and how much biomass 30 should be filled into the silo 97.

To the left of the silo 97, a feed system is provided for introducing flue gas 98 with low oxygen concentration. The feed system comprises a first valve 90 arranged and configured to regulate supply of flue gas 98 to the silo 97. The feed system comprises a second valve 90′ formed as a pressure reducing valve, which ensures that the silo 97 is pressurized with a pressure that is within a predefined range. Thus, an excess pressure (relative to the surroundings) is provided in the silo 97. This excess pressure prevents atmospheric air entering the silo 97. It is thus possible to reduce the oxygen concentration in the silo 97. This minimizes the oxygen concentration in the gas that is fed together with the biomass 30 into the reaction channel.

The silo outlet opens out into a screw channel in which there is a metering screw 92′ driven by an electric motor 100′. The activity (rotational speed) of the metering screw 92′ determines the amount of biomass the metering screw 92′ is metering per unit time.

A flap 99 is provided in the end of the housing in which the metering screw is arranged. The flap 99 is arranged and configured to open when biomass 30 is propelled forwards towards the flap 99. The biomass 30 that passes through the flap 99 drops down into a lower screw channel, which houses a feed screw 92, which is driven by an electric motor 100. The activity of the metering screw 92′ determines how much biomass 30 is fed into the reactor of the pyrolysis plant. The feed screw 92 is surrounded by a double walled jacket 95, which may be heated with hot pyrolysis gas 28 from a pipeline 142, which is the gas outlet from a filter system (not shown). In this way, the screw 92 and the biomass 30 that the feed screw 92 propels into the reactor is heated. The heating of the feed screw 92 may alternatively be provided with flue gas from burning of gas in the heating circuit.

FIG. 5B illustrates a schematic view of a production plant 10 according to an embodiment. The production plant 10 comprises a pyrolysis plant 1 according and an electrolyzer 44. The electrolyzer 44 produces hydrogen H₂ and oxygen O₂. The oxygen O₂, however, is a by-product derived from the manufacturing process of the electrolyzer 44. The electrolyzer 44 is connected to the pyrolysis plant 1 in a manner, in which oxygen O₂ from the electrolyzer 44 is used in the pyrolysis plant 1 and provided via an oxygen inlet.

In an embodiment, the production plant 10 comprises a Power-to-Gas or Power-to-Liquid plant 46 that is connected to and receives CO₂ that is stripped from gas from the reaction channel of the pyrolysis plant 1. The Power-to-Gas or Power-to-Liquid plant 46 carries out a methanol synthesis e.g. by the following reaction:

CO₂+3H₂↔CH₃OH+H₂O (1)

The Power-to-Gas or Power-to-Liquid plant 46 produces CH₃OH and receives hydrogen H₂ (e.g. from the electrolyzer 44) and CO₂ from the pyrolysis plant 1 or other sources.

In an embodiment, the Power-to-Gas or Power-to-Liquid plant 46 produces methanol through a microbial-based synthesis gas fermentation, in which a mixture of hydrogen, carbon monoxide, and carbon dioxide (known as syngas), is converted into fuel and chemicals.

### LIST OF REFERENCE NUMERALS

1 Pyrolysis plant
2 Reactor
3 Reaction channel
4 Outer wall
6 Feed section
8, 8′, 8″ Temperature sensor
10 Production plant
11 Flow direction
12 Control unit
14 Heating unit
16 Gas sensor
18 Heating circuit
20 Outlet
28 Pyrolysis gas
30 Biomass
40 Nozzle
41 Oxygen
42 CO₂
44 Electrolyzer
46 Power-to-Gas or Power-to-Liquid plant
48, 48′, 48″ Valve
48′″, 48″″ Valve
50, 52 Tank
54 Mixing chamber
56, 56′, 56″ Pipe
56′″, 56″″ Pipe
58 Graph
60 Graph
90, 90′ Valve
92 Feed screw
92′ Metering screw
95 Jacket
97 Silo
98 Flue gas
99 Flap
100′ Electric motor
102 Prior art reactor
104 Heat exchanger
103 Valve
103′ Valve
106 Upper inlet
105 Biochar
142 Pipeline
A, B, C Time period
Tₒₚₜᵢₘᵤₘ Predefined optimum temperature
Tᵤₚₚₑᵣ Predefined upper temperature
T_{lower} Predefined lower temperature
T Temperature
Q Flow

1. A pyrolysis plant comprising:
a reactor for producing pyrolysis gas from biomass, wherein the reactor comprises at least one reaction channel and at least one heating circuit, which is configured to heat the at least one reaction channel to a temperature that gasifies the biomass, wherein the reactor comprises a feed section configured for feeding the biomass into the at least one reaction channel, wherein the reaction channel comprises the heating circuit integrated in the reaction channel, wherein the heating circuit comprises a gas mixture unit and a plurality of input nozzles arranged and configured to introduce a mix of only oxygen and CO₂ from the gas mixture unit into the reaction channel,
at least one gas sensor arranged and configured to detect a concentration of oxygen (O₂) in the heating circuit;
a control unit; and
one or more temperature sensors,
wherein the one or more temperature sensors are arranged and configured to measure a temperature (T) in the reaction channel,
wherein the control unit is arranged and configured to regulate flow and/or oxygen concentration of the mix of oxygen and CO₂ into the reaction channel in dependency of the temperature (T) in the reaction channel.

2. The pyrolysis plant according to claim 1, wherein the control unit is configured to:
a) compare the temperature (T) in the reaction channel with a predefined temperature interval,
b) reduce the flow and/or the concentration of oxygen of the mixed gas introduced into the reaction channel if the temperature (T) in the reaction channel is above the predefined temperature interval, and
c) increase the flow and/or the concentration of oxygen of the mixed gas introduced into the reaction channel if the temperature (T) in the reaction channel is below the predefined temperature interval.

3. The pyrolysis plant according to claim 1, further comprising a heating unit arranged and configured to heat the mix of oxygen and CO₂ before the mix enters the reaction channel.

4. The pyrolysis plant according to claim 1, further comprising an outlet arranged and configured to move gas out of the reaction channel.

5. A production plant comprising the pyrolysis plant according to claim 1 and an electrolyzer, wherein the electrolyzer is connected to the pyrolysis plant such that oxygen (O₂) from the electrolyzer is provided to the pyrolysis plant via an oxygen inlet.

6. The production plant according to claim 5, further comprising a Power-to-Gas or Power-to-Liquid plant, wherein the Power-to-Gas or Power-to-Liquid plant is connected to and receives CO₂ that is stripped from gas from the reaction channel of the pyrolysis plant.

7. A method for producing pyrolysis gas from biomass in the pyrolysis plant of claim 1, the method comprising the following steps:
heating the reaction channel by introducing the mix of only oxygen and CO₂ into the reaction channel;
detecting the concentration of oxygen (O₂) in the heating circuit:
detecting the temperature (T) in the reaction channel; and
regulating the flow of the mix of oxygen and CO₂ into the reaction channel in dependency of the temperature (T) in the reaction channel.

8. The method according to claim 7, further comprising the steps of:
a) comparing the temperature (T) in the reaction channel with a predefined temperature interval,
b) reducing the flow and/or the concentration of oxygen of the mixed gas introduced into the reaction channel if the temperature (T) in the reaction channel is above the predefined temperature interval, and
c) increasing the flow and/or the concentration of oxygen of the mixed gas introduced into the reaction channel if the temperature (T) in the reaction channel is below the predefined temperature interval.

9. The method according to claim 7, further comprising the step of heating the mix of oxygen and the CO₂ before the mix enters the reaction channel.

10. The method according to claim 7, further comprising the step of moving the CO₂ out of gas from the reaction channel.
